(19) United States
(12) Patent Application Publication
(10) Pub. No.: US 2022/0386899 A1
(43) Pub. Date: Dec. 8, 2022

(54) Wearable device to identify medical emergencies and notify

(71) Applicant: Mirza Faizan, Irving, TX (US)

(72) Inventors: Mirza Faizan, Irving, TX (US); Mirza Rizwan, Patna (IN); Danish Khan, Murphy, TX (US); Bilal Mohammed Majid, Plano, TX (US); Mikhail Imtiaz, Carrollton, TX (US); Sama Fatima, Carrollton, TX (US); Roshan Shaik, Plano, TX (US); Ayush Bhattacharya, Frisco, TX (US); Umar Mohammed, Plano, TX (US); Sidra Ambreen, Plano, TX (US); Areebah Mohiuddin, Plano, TX (US); Maham Abbasi, Irving, TX (US)

(21) Appl. No.: 17/339,327

(22) Filed: Jun. 4, 2021

(51) Int. Cl. A61B 5/11; A61B 5/00; G16H 10/60

(52) U.S. Cl. CPC A61B 5/1117; A61B 5/7455; A61B 5/7405; A61B 5/0022; A61B 5/002; A61B 5/0004; A61B 5/681; A61B 5/1112; A61B 5/747; G16H 10/60; A61B 2560/04

(57) ABSTRACT

An electronic battery-operated medical bracelet device that contains the technology to accurately detect collapse/fall or other medical emergencies, through the monitoring of the heart rate, oxygen saturation, heart rhythm, and patient input. The bracelet will also display the vital readings to the user if the readings return as abnormal, in an attempt to persuade the user to seek medical attention before it becomes worse. Once a medical emergency is detected, the device alerts surrounding people and paramedics of the emergency and distributes medical and personal information different for surrounding people and paramedics. This includes medical conditions, health insurance information, next of kin, etc. This information can be uploaded to the device's onboard memory drive and can share the information or steps to reach the information, through an onboard speaker system, Bluetooth pairing, and near-filled communication (NFC) to which paramedics have special access.

### TECHNICAL FIELD

The invention relates to a wearable emergency medical device. Further, as the time taken to diagnose pre-existing medical condition(s) when a person collapses from a medical emergency before treatment may take too long, and puts the person at risk of dying prematurely. More specifically, this device can be worn by the user as the medical bracelet device which will constantly monitor the vital signs comprising of the heart rate and blood oxygen saturation in real time, and record the readings.

### BACKGROUND OF THE INVENTION

In the global era, holiday travel is very common, and many of them have medical conditions that could prove to be deadly if untreated for long enough.

It has been very difficult to identify travelers in foreign countries, and their medical history and conditions. When a person collapses, the time it takes to diagnose his/her pre-existing medical condition(s) before treatment may take too long and puts the user at risk of dying prematurely. This can be attributed to the fact that foreign paramedics and medical professionals require multiple diagnoses and tests to identify the patient's medical conditions as they do not have immediate access to the patient's medical history.

Most collapses/falls from a medical emergency (such as cardiac arrest, heart attacks, Chronic Obstructive Pulmonary Disease or COPD, etc.) can be attributed to hypoxia, a lack of oxygen to the brain. In other words, the emergency becomes at risk of being deadly if 1) the brain doesn't get enough blood, or 2) the blood reaching the brain has low oxygen saturation levels.

The available products in the market can monitor vital signs through a wearable device, but lack the ability to automatically detect a medical emergency, and automatically send that information wirelessly to others, instead depending on human input for every major step, proving to be problematic when the person cannot physically interact with the device. It also cannot store medical information about the patient, making an overseas diagnosis, and basic first aid difficult. Time is needed for these crucial parts of data, and unnecessary tests put the patient at risk of premature death.

Conventionally available devices in market does not contain the capacity to monitor a user vital signs for health problems, instead utilizes onboard sensors for fall detection. The prior arts ability to identify medical emergencies is severely hampered because of this limitation. Furthermore, prior art does not retain the ability to store vital signs and medical history. A connection to a mobile phone is required in this prior art in order to contact emergency services.

Thus, there is an unmet need for a device which can solve the above mentioned drawback and to tackle this issue, the device will monitor the heart rate, and blood oxygen saturation levels, as the primary methods to differentiate between a medical emergency and standard conditions.

### SUMMARY OF THE INVENTION

The present invention discloses a wearable medical emergency detection device. The detection device comprising a memory unit, a processor, and an electronic unit. Further, the electronic unit comprising a plurality of sensors. The plurality of sensors are placed at plurality of locations, wherein the plurality of sensors are further configured to monitor information of a user. The monitored information may include at least a plurality of vitals of the user. The device further includes an information sharing unit which may be configured to transmit the detected information. The device further includes an alarming unit which may be configured to notify an alarm in case of detected emergency based on the detected information, and may also include an audio unit configured for receiving one or more voice enabled communications from the notified user to the user wearing the medical emergency detection system.

In an embodiment of the present invention, a wearable medical emergency detection device is disclosed. The detection device comprising a memory unit; a processor which may be configured to trigger an electronic unit. The triggering is based on a sudden change in a position of a user. The position may correspond to a standing position or a fallen position on the ground. The electronic unit further comprises a plurality of sensors which may be configured to monitor a plurality of vitals of a user. The vitals may include at least a blood pressure, a heart rate, an Electrocardiogram reading, and a blood oxygen level. The device further comprises an information sharing unit which may be configured to transmit the monitored plurality of vitals. The device may further include an alarming unit which may be configured to notify in case of detected emergency based on an abnormal condition detected based on the transmitted plurality of vitals of the user.

In an embodiment of the present invention, a method to detect a medical emergency by a wearable medical emergency detection device is disclosed. The method may comprise monitoring information of a user. The information includes at least vitals of the user. The method further includes transmitting to a plurality of other users the monitored information of the user. Further, notifying in case of detected emergency based on the transmitted information. The notification is communicatively transmitted to a plurality of contacts and furthermore the method includes generating an alarm based on which a plurality of nearby people can help the user under emergency.

### OBJECTIVE OF INVENTION

The objective of the disclosed invention is to have the ability to identify medical emergencies.

Yet another objective of the invention is to store the medical history of a patient in the event of an emergency in the device. The device is designed to monitor vital signs with an attempt of conducting a diagnosis. It includes an accelerometer and gyroscope to detect a user's fall in the event of a medical emergency.

Yet another objective of the present invention is to monitor a user's vital signs, either through an electrocardiogram or a pulse oximeter.

Yet another objective of the present invention is that if a user is in need of medical attention this will notify paramedics or surrounding people.

Yet another objective of the invention is to communicatively transmit the medical records, and measured vitals in case of abnormal activity to emergency contacts and the medical staff.

Yet another objective of the present invention is to instruct medical team to access the medical history of the user in emergency, so that right treatment is started immediately.

### BRIEF DESCRIPTION OF THE DRAWINGS

The accompanying drawings illustrate various embodiments of systems, methods, and other aspects of the disclosure. Any person having ordinary skill in the art will appreciate that the illustrated element boundaries (e.g., boxes, groups of boxes, or other shapes) in the figures represent one example of the boundaries. It may be that in some examples, one element may be designed as multiple elements or that multiple elements may be designed as one element. In some examples, an element shown as an internal component of one element may be implemented as an external component in another, and vice versa. Furthermore, elements may not be drawn to scale.

Various embodiments will hereinafter be described in accordance with the appended drawings, which are provided to illustrate, and not to limit the scope in any manner, wherein like designations denote similar elements, and in which:

FIG. 1A shows an Isometric view of the bracelet along with the majority of necessary components to the bracelet;

FIG. 1B is another isometric view of the bracelet to specifically show the hidden features;

FIG. 2 is a block diagram illustrating a system environment in which various embodiments may be implemented;

FIGS. 3a and 3b collectively is a flowchart illustrating a method to identify medical emergencies and notify, in accordance with at least one embodiment; and

### DETAILED DESCRIPTION OF DRAWINGS

The present disclosure is best understood with reference to the detailed figures and description set forth herein. Various embodiments are discussed below with reference to the figures. However, those skilled in the art will readily appreciate that the detailed descriptions given herein with respect to the figures are simply for explanatory purposes as the methods and systems may extend beyond the described embodiments. For example, the teachings presented and the needs of a particular application may yield multiple alternate and suitable approaches to implement the functionality of any detail described herein. Therefore, any approach may extend beyond the particular implementation choices in the following embodiments described and shown.

References to “one embodiment,” “an embodiment,” “at least one embodiment,” “one example,” “an example,” “for example,” and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in an embodiment” does not necessarily refer to the same embodiment.

### Definitions

The following terms shall have, for the purposes of this application, the respective meanings set forth below.

A “computing device” refers to a device that includes one or more processors/microcontrollers and/or any other electronic components, or a device or a system that performs one or more operations according to one or more programming instructions/codes. Examples of a computing device may include, but are not limited to, a desktop computer, a laptop, a personal digital assistant (PDA), a mobile device, a smartphone, a tablet computer (e.g., iPad®, and Samsung Galaxy Tab®), and the like.

A “patient or a user” is a human being who may require medical care or treatment by a medical expert, such as a doctor. In other words, a patient is a recipient of health care services provided by a health practitioner. In an embodiment, a patient refers to a patient who is currently under medical observation.

An “electronic medical record” refers to a documentation of health condition of a patient. In an embodiment, the medical record may include periodic measures of physiological parameters associated with the patient. Further, the medical record may include nursing notes documented over a specific time by a healthcare professional (such as a doctor, a nurse, a medical attender, a hospital staff, and/or the like). In an embodiment, the nursing notes may include recorded observations, administered drugs and therapies, test results, X-rays, nursing reports, investigative reports, and the like. In an embodiment, the medical record may be documented on a computing device, such as, but not limited to, a desktop computer, a laptop, a PDA, a mobile device, a smartphone, a tablet computer (e.g., iPad® and Samsung Galaxy Tab®), and the like. In an embodiment, the medical record may correspond to electronic or handwritten document(s).

A “nursing note of vitals” refers to a medical record that may describe a health condition of a patient and an administered or planned treatment. The nursing note may be documented by a nurse, physician, and other healthcare professionals for recording the health condition of the patient. The nursing note may comprise prescribed treatments, response to the prescribed treatments, medical diagnosis, and/or the like. The nursing note, corresponding to the patient, may be recorded on a daily or periodic basis. Hereinafter, “nursing note” and “vitals report” may be interchangeably used.

“Historical data” refers to one or more medical records or vitals of one or more users who were under medical observations in the past. In an embodiment, the one or more medical records may comprise a measure of one or more physiological parameters (e.g., blood pressure, heart rate, respiratory rate, body temperature, and the like) associated with the one or more patients. Further, the one or more medical records may comprise lab investigation data (e.g., a sodium level, a potassium level, a glucose level, and the like), diagnostics data, and other medical data associated with the one or more patients.

A “sensor” refers to a device that detects/measures events or changes in quantities and provides a corresponding output, generally as an electrical or optical signal. In healthcare domain, a first type of sensors may be operable to detect and measure various biological and physical variations corresponding to the patient. Such detected and measured signals may be recorded for further analytics. For example, biomedical sensors are used to monitor heart rate, respiration rate, pulse rate, blood pressure, and the like, of the first patient. Further, sensors may be operable to detect and measure various physical and/or chemical signals corresponding to a medical device associated with the patient. For example, pressure sensors, temperature sensors, and humidity sensors are used to monitor and regulate gas flow and gas conditions in anesthesia machines, respirators, and ventilators. The sensor may be an acceleration sensor or a vibration sensor, such as a VTT or TI standard chip base accelerometer. These examples are currently contemplated, but it should be understood that alternatives exist.

A “wearable device” refers to a device which may be easily worn by a user. The user may wear the wearable device at a plurality of body parts and not necessarily a wrist of the user.

A “fall sensor” refers to a fall detection sensor. In an embodiment, a fall sensor is responsive to a physical effect of a fall, whether measured by vibration, shock, acceleration, sound, a combination of the foregoing effects, or some other measurable physical effect of a fall as determined to be desirable. These may vary according to the intended use, the intended location, or the expected risk to an end-user wearing the bracelet. Also, more than one type of fall sensor may be incorporated in bracelet to sense more than one physical effect of a fall. A gyroscope or accelerometer may also be used alternatively in place of the fall sensor.

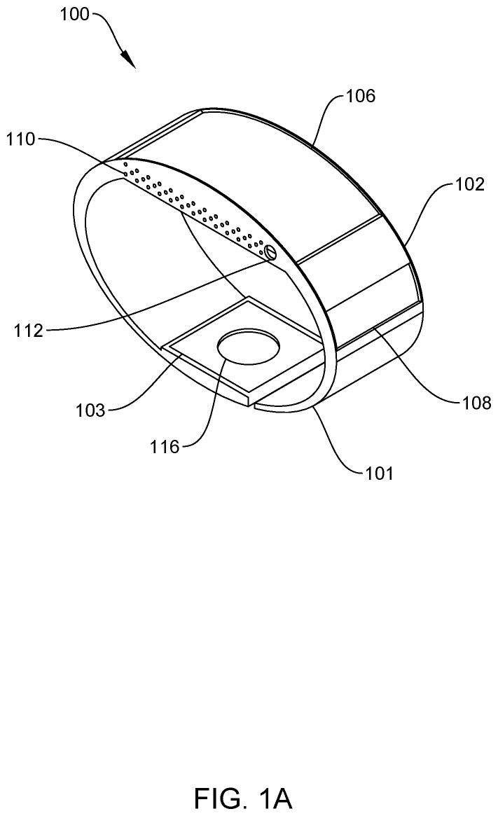

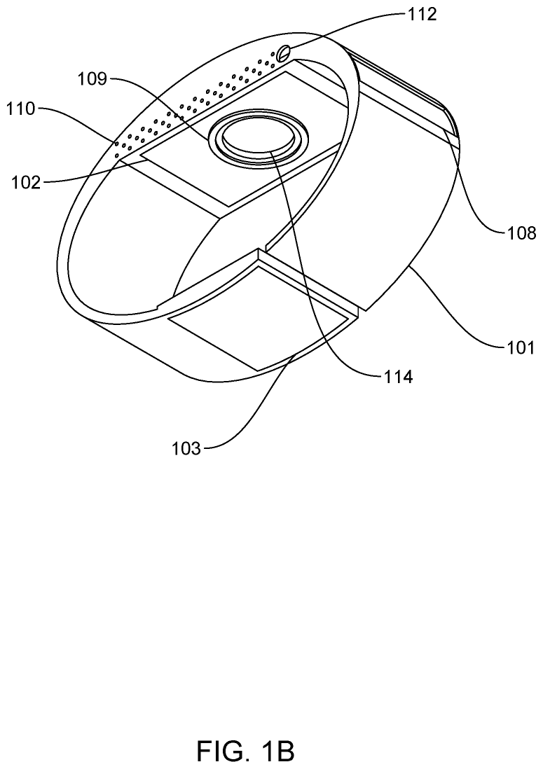

FIG. 1A illustrates a wearable medical emergency detection device 100 in which various embodiments may be implemented.

Referring first to FIG. 1A, a wearable medical emergency detection device 100 is shown in exemplary form. Wearable medical emergency detection device 100 is illustrated as a bracelet. In an embodiment the wearable device may be worn on the wrist. The bracelet consists of a wristband 101 that has two main pieces of hardware, the hardware housing unit 102, and the photo sensor housing unit 103. Alternate examples of wearable device forms include, but are not limited to, necklaces, rings, pin-on items, belts, watches, belt attachments, chest bands, items capable of being carried in a pocket, and articles of clothing.

In an embodiment of the present invention, a top face of the hardware housing unit 102 of the wearable medical emergency detection device 100 may be made of a touch screen 106. In an embodiment the touch screen may be configured to display one or more information such as real time data, vitals, and the alike.

In a further embodiment of the present invention, the touch screen may also display one or more notifications, such notification may include an emergency notification, an incoming message notification.

In an embodiment, the hardware housing unit 102 may include first of two Electrocardiogram (ECG) nodes 108 that may be located beneath the touch screen sensor of the touch screen 106. Furthermore, a second ECG node 109 (shown in FIG. 1B) that may be located on the bottom of the hardware housing unit 102 of the wearable medical emergency detection device 100.

In an embodiment, the hardware housing unit 102 of the wearable medical emergency detection device 100 may include a plurality of speakers 110 on both of its sides. The left side of the speaker 110 may have an audio jack 112 for the user to plug in headphones. Device 100 may also communicate audibly using audio codec, which may receive spoken information from a user and convert it to usable digital information. Audio codec may likewise generate audible sound for a user, such as through a speaker, e.g., in a handset of device 100. Such sound may include sound from voice telephone calls, may include recorded sound (e.g., voice messages, music files, etc.) and may also include sound generated by applications operating on device 100.

In an embodiment, the hardware housing unit 102 may include a power unit. The power unit may include one or more batteries. The one or more batteries may be solar enabled chargeable batteries. The chargeable batteries may provide power of each and every unit of the wearable medical emergency detection device 100.

In an embodiment, the hardware housing unit 102 of the wearable medical emergency detection device 100 may include a plurality of sensors. The plurality of sensors may include accelerometer/gyroscope that may be configured to detect falls.

In an embodiment, the hardware housing unit 102 may include a memory chip to store information. The memory may stores information within the device 100. In one implementation, the memory is a volatile memory unit or units. In another implementation, the memory is a non-volatile memory unit or units. The memory may also be another form of computer-readable medium, such as a magnetic or optical disk. The memory may store the medical records, personal data of the user.

The memory is capable of providing mass storage for the device 100. In one implementation, the memory may be or contain a computer-readable medium, such as a floppy disk device, a hard disk device, an optical disk device, a tape device, a flash memory or other similar solid state memory device, or an array of devices, including devices in a storage area network or other configurations.

In an embodiment, the hardware housing unit 102 may include a Bluetooth transmitter and receiver to send and receive limited amounts of information. Device 100 may communicate wirelessly through communication interface, which may include digital signal processing circuitry where necessary. Communication interface may provide for communications under various modes or protocols, such as GSM voice calls, SMS, EMS, or MMS messaging, CDMA, TDMA, PDC, WCDMA, CDMA2000, or GPRS, among others.

Such communication may occur, for example, through radio-frequency transceiver. In addition, short-range communication may occur, such as using a Bluetooth, Wi-Fi, or other such transceiver (not shown). In addition, GPS (Global Positioning System) receiver module may provide additional navigation- and location-related wireless data to device 100, which may be used as appropriate by applications running on device 100.

In an embodiment, the hardware housing unit 102 may include a NFC chip to send all information, GPS system to find location. In an alternate embodiment, the hardware housing unit 102 may be communicatively connected with the wireless communication technology.

In an embodiment, the hardware housing unit 102 may include a micro processing unit to act on given values, is located inside the hardware housing unit 102. The micro processing unit may be configured to determine various colors of emergency as yellow, red based on the computation of values of various determined vitals from the user.

In an embodiment, the device includes a photo sensor housing unit 103. The housing unit may include one or more photo sensors 116. The photo sensors may be configured to determine the generated light by receiver and transmitter connection.

FIG. 1 B illustrates an enhanced wearable medical emergency detection device 100 in which various embodiments may be implemented.

The FIG. 1B discloses that the device my further include a light emitting diode 114. The light emitting diode may be used in conjunction with the one or more photo sensors 116 (shown in FIG. 1A) to create pulse oximeter via a cross section of the wrist. The device may further include an electrocardiogram ECG node.

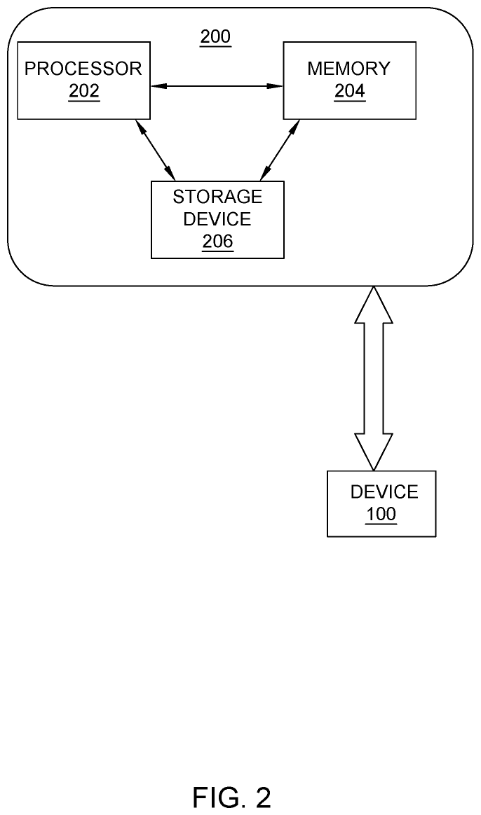

FIG. 2 shows an example of a computing device 200 and a device 100 that can be used to implement the techniques described here. Computing device 200 is intended to represent various forms of digital computers, such as laptops, desktops, workstations, personal digital assistants, servers, blade servers, mainframes, and other appropriate computers. Computing device 200 is intended to represent various forms of mobile devices, such as personal digital assistants, cellular telephones, smartphones, and other similar computing devices. The components shown here, their connections and relationships, and their functions, are meant to be exemplary only, and are not meant to limit implementations described and/or claimed in this document.

Computing device 200 includes a processor 202, memory 204, a storage device 206, a high-speed interface connecting to memory 204 and high-speed expansion ports, and a low speed interface connecting to low speed bus and storage device 206. Each of the components 202, 204, 206 are interconnected using various busses, and may be mounted on a common motherboard or in other manners as appropriate. The processor 202 can process instructions for execution within the computing device 200, including instructions stored in the memory 204 or on the storage device 206 to display graphical information for a GUI on an external input/output device, such as display coupled to high speed interface. In other implementations, multiple processors and/or multiple busses may be used, as appropriate, along with multiple memories and types of memory. Also, multiple computing devices 200 may be connected, with each device providing portions of the necessary operations (e.g., as a server bank, a group of blade servers, or a multi-processor system).

The memory 204 stores information within the computing device 200. In one implementation, the memory 204 is a volatile memory unit or units. In another implementation, the memory 204 is a non-volatile memory unit or units. The memory 204 may also be another form of computer-readable medium, such as a magnetic or optical disk. The memory may also be a part of hardware housing unit 102.

The storage device 206 is capable of providing mass storage for the computing device 500. In one implementation, the storage device 206 may be or contain a computer-readable medium, such as a floppy disk device, a hard disk device, an optical disk device, a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described above. The information carrier is a computer- or machine-readable medium, such as the memory 204, the storage device 206, or memory on processor 202.

The high-speed controller manages bandwidth-intensive operations for the computing device 200, while the low speed controller manages lower bandwidth-intensive operations. Such allocation of functions is exemplary only. In one implementation, the high-speed controller is coupled to memory 204, display (e.g., through a graphics processor or accelerator), and to high-speed expansion ports, which may accept various expansion cards (not shown). In the implementation, low-speed controller is coupled to storage device 206 and low-speed expansion port. The low-speed expansion port, which may include various communication ports (e.g., USB, Bluetooth, Ethernet, wireless Ethernet), may be coupled to one or more input/output devices, such as a keyboard, a pointing device, a scanner, or a networking device such as a switch or router, e.g., through a network adapter.

The computing device 200 may be implemented in a number of different forms, as shown in the figure. For example, it may be implemented as a standard server, or multiple times in a group of such servers. It may also be implemented as part of a rack server system. In addition, it may be implemented in a personal computer such as a laptop computer. Alternatively, components from computing device 200 may be combined with other components in a mobile device (not shown), such as device 100. Each of such devices may contain one or more of computing device 200, and an entire system may be made up of multiple computing devices 100, 200 communicating with each other.

The processor 202 can execute instructions within the computing device 200, including instructions stored in the memory 204. The processor may be implemented as a chipset of chips that include separate and multiple analog and digital processors.

Processor 202 may communicate with a user through control interface and display interface coupled to a display. The display may be, for example, a TFT LCD (Thin-Film-Transistor Liquid Crystal Display) or an OLED (Organic Light Emitting Diode) display, or other appropriate display technology. The display interface may comprise appropriate circuitry for driving the display to present graphical and other information to a user. The control interface may receive commands from a user and convert them for submission to the processor 202. In addition, an external interface may be provided in communication with processor 202, so as to enable near area communication of device 100 with other devices. External interface may provide, for example, for wired communication in some implementations, or for wireless communication in other implementations, and multiple interfaces may also be used.

The memory stores information within the computing device 200 or 100. The memory can be implemented as one or more of a computer-readable medium or media, a volatile memory unit or units, or a non-volatile memory unit or units. Expansion memory may also be provided and connected to device 200 through expansion interface, which may include, for example, a SIMM (Single In Line Memory Module) card interface. Such expansion memory may provide extra storage space for device 200/100, or may also store applications or other information for device 100/200. Specifically, expansion memory may include instructions to carry out or supplement the processes described above, and may include secure information also. Thus, for example, expansion memory may be provide as a security module for device 100, and may be programmed with instructions that permit secure use of device 100. In addition, secure applications may be provided via the SIMM cards, along with additional information, such as placing identifying information on the SIMM card in a non-hackable manner.

The memory may include, for example, flash memory and/or Non Volatile RAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory, expansion memory, or memory on processor.

Device 100 may communicate wirelessly through communication interface, which may include digital signal processing circuitry where necessary. Communication interface may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver. In addition, short-range communication may occur, such as using a Bluetooth, Wi-Fi, or other such transceiver (not shown). In addition, GPS (Global Positioning System) receiver module may provide additional navigation- and location-related wireless data to device 100, which may be used as appropriate by applications running on device 100.

Device 100 may also communicate audibly using audio codec, which may receive spoken information from a user and convert it to usable digital information. Audio codec may likewise generate audible sound for a user, such as through a speaker, e.g., in a handset of device 100. Such sound may include sound from voice telephone calls, may include recorded sound (e.g., voice messages, music files, etc.) and may also include sound generated by applications operating on device 100.

The computing device 200 may be implemented in a number of different forms, as shown in the figure. For example, it may be implemented as a cellular telephone. It may also be implemented as part of a smartphone, personal digital assistant, or other similar mobile device.

Additionally computing device 200 or device 100 can include Universal Serial Bus (USB) flash drives. The USB flash drives may store operating systems and other applications. The USB flash drives can include input/output components, such as a wireless transmitter or USB connector that may be inserted into a USB port of another computing device.

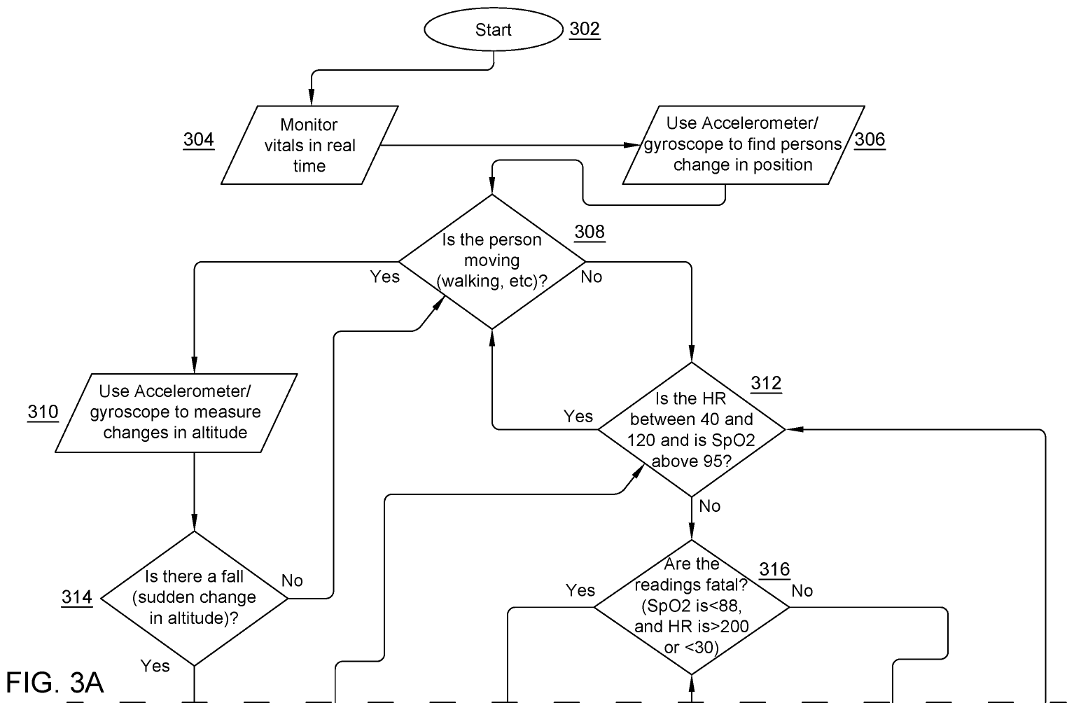

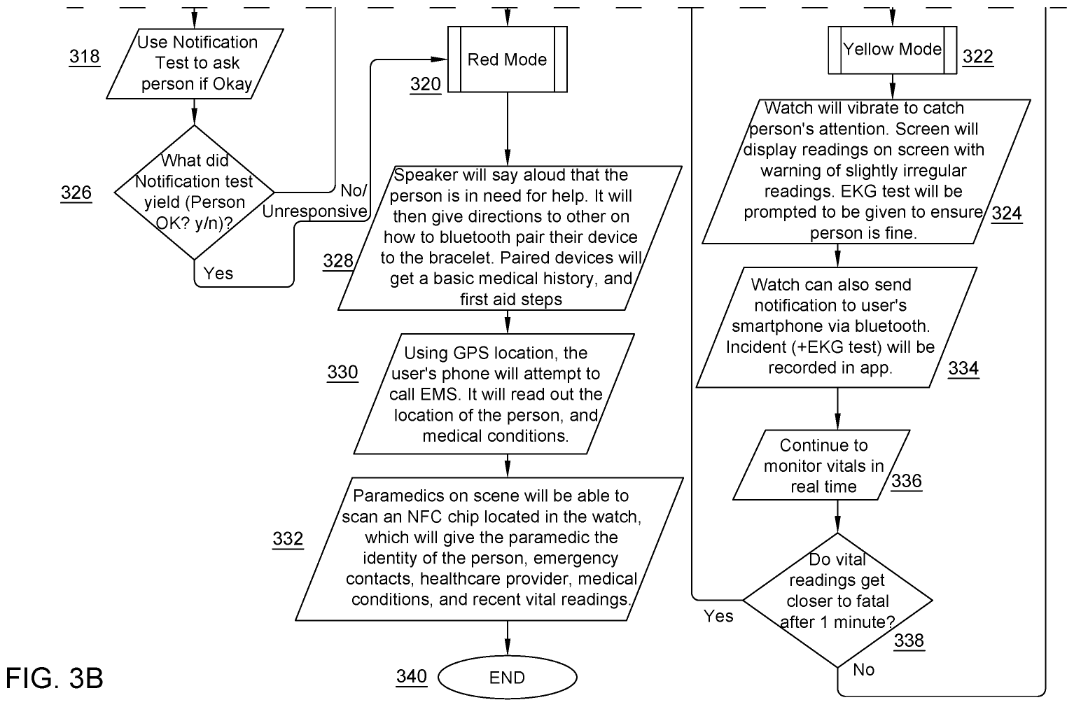

FIGS. 3a and 3b collectively is a flowchart illustrating a method to identify medical emergencies and notify, in accordance with at least one embodiment. With reference to FIGS. 3a and 3b, there is shown a flowchart 300 that is described in conjunction with FIGS. 1A, and 1B. The method starts at step 302 and proceeds to step 304.

At step 304, vitals of the user are monitored in real time. In an embodiment, the hardware housing unit 102 may be configured to monitor the vitals of the user. In an embodiment, a plurality of sensors equipped on the hardware housing unit 102 of the device may be configured to measure the plurality of the vitals of the user.

The plurality of vitals may include Body temperature, Pulse rate, Respiration rate (rate of breathing), and Blood pressure. The vitals recited here are just for exemplary usage and are not limited.

At step 306, a change in position of the user is detected by the plurality of sensors. In an embodiment, the plurality of sensors for detecting the change in the position of the user may be an accelerometer. In an alternate embodiment, the change in the position of the user may be detected by a gyroscope.

At step 308, a check is performed to determine whether the user is walking or not. In an embodiment, the plurality of sensors may be operable to perform the check to determine whether the user is walking or not. In an embodiment, if the sensor determines that the user is walking then the control passes to step 310. In an embodiment, if the sensor determines that the user is not walking then the control passes to step 312.

At step 310, a change in an altitude is measured by the plurality of sensors. In an embodiment, the plurality of sensors may be configured to measure the change in altitude of the user. In an embodiment, the sensor may be accelerometer. In an alternate embodiment, the sensor may be a gyroscope. The step then jumps to step 314.

At step 312, a check is performed to determine that a HR is between 40 and 120, and SₚO2 above 95. In an embodiment, the processor may be configured to determine that the HR is between 40 and 120, and SₚO2 above 95. If it is yes, then step moves back to step 308. If it is No, then the step moves to 316.

At step 314, check is performed that a fall is occurred of the user. In an embodiment, the plurality of sensors may be configured to detect the fall of the user by the change in the altitude. In an embodiment, the sensor may be accelerometer. In an alternate embodiment, the sensor may be a gyroscope. In an embodiment, using a technology known as actigraphy, the accelerometer and gyroscope may detect whether the person is moving or if they fall, being detected through a sudden shift in altitude. If it is yes, then a step 318 is executed and if no, then the step 308 may be executed.

At step 316, a check is performed to determine that the HR is lower than 200 and greater than 30, and SₚO2 is above 88. In an embodiment, the processor may be configured to determine that the HR is lower than 200 and greater than 30, and SₚO2 is above 88. If it is yes, then step moves to step 320. If it is No, then the step moves to 322.

At step 320, a condition is marked as a red mode. In an embodiment, the processor may be configured to mark the condition as the red mode. The red mode may be considered as a high risk mode.

At step 322, a condition is marked as a yellow mode. In an embodiment, the processor may be configured to mark the condition as the yellow mode. The process jumps to step 324.

At step 318, a use notification is tested to ask if the user is in well condition. In an embodiment the processor is configured to test the notification that if the user is in well condition.

At step 326, a notification output from the user is determined. In an embodiment, the processor is configured to determine the output result of the user for the given notification test. This is where it will ask the user if they need medical assistance. If the person responds with ‘YES’, the bracelet will automatically skip the tests for other vital signs and alert the people around them and notify paramedics. If the response is a ‘NO’, or if there is no response at all, the bracelet will continue with the readings of vital signs to ensure that the person is in fact not needing medical attention. Hence, If the notification test is yes, then the system jumps to step 320 or if no, then the system jumps to 312.

At step 328, a speaker associated with the system is activated. The speaker may be required to notify nearby people regarding help requirement by the user. In an embodiment, the processor may be configured to detect that the user requires an immediate attention and the speaker may generate a loud sound for the attention of the nearby people around the user.

In an embodiment, the processor may be configured to direct the nearby people on how to pair the bluetooth of the medical device with the Bluetooth of the respective phone devices of the nearby people.

In a further embodiment, after the devices are paired, then the nearby people may retract the historical data or the medical data of the user. Furthermore, the medical aiding steps may also be retracted from the connected device.

At step 330, a location of the user is determined. The location of the user may be determined by the GPS locator. In an embodiment, the GPS system may be enabled to allow the medical teams to locate the user.

In an embodiment, the user's device may try and communicate with EMS or the emergency medical system. Upon communication, the user's location and medical data may be transmitted to the EMS.

At step 332, a NFC chip located in the device of the user is scanned by one or more scanning devices held with the one or more medical staff. The processor may be configured to facilitate the data transmission. In an embodiment, the NFC chip may include data of the user, such as but not limited to the user's identity, one or more emergency contacts of the user, medical conditions of the user, and/or recent one or more vital conditions of the user. The process ends at Step 340.

At step 324, vibrations are provided by the device to the user. Further, the screen of the device will display the one or more readings or the vitals generating due to movement of the user arising due to the vibrations provided to the user.

In an embodiment, an ECG (EKG) test may also be performed simultaneously with the step 324.

At step 334, one or more notifications are sent from the device to the user's watch. The processor may be configured to send one or more notifications from the device to the user's watch. In an embodiment, the one or more notifications may include notifications of health update and EKG test. In an embodiment, the memory of the device may be configured to store the notifications of the health update and reports of the EKG test.

At step 336, one or more vitals of the user are monitored continuously. The vitals may be monitored by one or more sensors of the device. In an embodiment, the one or more sensors along with the processor may be configured to monitor one or more vitals of the user.

At step 338, a check is performed that if one or more vitals of the users are closer to the pre determined one or more vitals. The one or more pre determined vitals may be stored in the memory of the device. In an alternate embodiment, the one or more pre-determined vitals may be based on a plurality of attributes, such as age, gender, location, and medical history of the user. If the result is no, then the step jumps to 312. If it is yes, then the step jumps to 316.

The process ends at step 340.

In an exemplary embodiment, the entire process 300 is explained with example parameters set for Heart Rate (HR) and Blood Oxygen Concentration (SpO2). By utilizing the real time monitoring of the onboard pulse oximeter, the bracelet or the medical device may go through a series of decisions based on the vitals' data being received from the user.

Once worn by the user, the medical bracelet may constantly monitor the vital signs comprising of the heart rate and blood oxygen saturation in real time, and record the readings. The configuration of the device includes a pulse oximeter that may get the readings through the cross section of the user's wrist. This embodiment may be a necklace, elbow brace, socks, knee brace and a groin cup. The technology used to monitor the heart rate and blood oxygen levels is called photoplethysmography and is based on the principle of flashing different colors of light in order to determine the heart-beats per minute, and blood oxygen saturation. These readings may determine the overall health of the user.

The device may be further equipped with both an accelerometer and gyroscope, which are both capable of detecting the user's change in position or altitude in space, for when the user is on move. Using a technology known as actigraphy, the accelerometer and gyroscope may detect whether the person is moving or if they fall, being detected through a sudden shift in altitude. The embodiment of this could be a watch, phone, footwear, jewellery, sunglasses, headband, headphones, clothes, bags, makeup equipment and accessories. Meanwhile, the bracelet may continue to monitor and record the vital signs in real time.

When a fall is detected, the bracelet may enact the notification test, which may be displayed on the bracelet screen or on the user's phone. This is where it will ask the user if they need medical assistance. If the person responds with ‘YES’, the bracelet will automatically skip the tests for other vital signs and alert the people around them and notify paramedics. If the response is a ‘NO’, or if there is no response at all, the bracelet will continue with the readings of vital signs to ensure that the person is in fact not needing medical attention.

If the pulse oximeter detects that the person does need medical attention, the bracelet will begin a series of warnings by the colors Green, Yellow and Red to persuade the user to get medical help. If it's in the green zone, the user is safe and does not need any medical help. If it's in the yellow zone, the user's vital signs are abnormal, but not fatal and may return to normal.

In yellow mode, the bracelet will display the readings on the screen, advising the user to see a doctor or medical professional in the near future, while constantly checking on the user to see if the readings move closer to fatal (in which red mode will be activated). If it's in the red zone, where the readings are fatal or dangerous to health, the user is in need of immediate medical help.

However, if there is no fall, the bracelet will constantly be reading the vital signs. When the bracelet encounters abnormal results, it will go into yellow mode, and if the readings move to fatal, it will enter red mode. An electrocardiogram test may be given to the patient in yellow mode if the heart rate indicates some kind of emergency and if the blood oxygen levels indicate that the person is fine and conscience.

When the medical emergency has positivity been identified (i.e. red mode), the bracelet will distribute the call for help, and allow the patient's medical information (stored in the memory drive) to be distributed to surrounding people and paramedics. A speaker system onboard the bracelet will say aloud that the person is in need for help and will then give directions to others on how to bluetooth pair their device to the bracelet. Paired devices will get a basic medical history, and first aid steps.

Further, if the bracelet is connected through Bluetooth connection to the patient's phone, it can automatically call emergency services, and read off the patient's medical information, as well as approximate location (taken through the bracelet's GPS system). The bracelet will also send out messages to everyone in close proximity through Bluetooth sharing capabilities, which will display a very basic version of the patient's medical history and how to perform first aid on the patient.

The specific amount of information that will be shared can be pre-determined by the patient. Once paramedics arrive, the NFC chip within the bracelet will transfer the patient's personal information, medical conditions, doctor's name along with his/her phone number, health insurance information, next of kin, etc, through a special device carried by the paramedics (to ensure no random passer-by has access to the information). Using these functions, the bracelet will allow the patient to receive proper treatment at a much faster rate and will decrease his or her chances of dying prematurely.

In an alternate embodiment, the Bracelet may be made from different materials. The different materials may be a combination of metal and polymer materials. The bracelet may be adjustable with an open portion adapted to fit on one or more inner portions of a user's wrist. Other materials and combinations of materials, including precious metals and gems so that the bracelet functions as jewelry. Some examples may be designed for style and/or fashion to have a subjectively attractive external appearance. Furthermore, the Bracelet may be flexible and/or may be abrasion resistant and/or UV stable.

In a further embodiment, the functional components of bracelet are illustrated as being embedded in or otherwise integrated into the bracelet, other methods for securing or attaching some or all of the components should be possible, including external attachments and making the body of bracelet hollow to permit internal mounting of components.

In an embodiment, the device may include a transmitter that may be a standard use 915 mHz, 2.4 GHz, or 6.0 Ghz transmitter of any type commonly used in wireless phone applications.

A chipset firmware can be suitably programmed or encoded to coordinate the interaction of fall sensor and transmitter. While a chipset is illustrated as the device for effecting and controlling the functionality of bracelet, alternatives and equivalents including, but not limited to, other types of controller, software, hardware, and/or firmware may be suitable.

In an exemplary embodiment a fall event may be sensed by the fall sensor in various manners and by various methods. For example, fall sensor may measure the movement activity of a user and determine that a fall event has occurred by the relative measurements of activity, recognizing a fall upon sensing a relatively large impact user activity followed by a period of substantial inactivity, no activity, or substantially reduced activity. Other fall and/or activity measurements are possible by fall sensor to determine the incident of a fall event. For example, a settle time after impact of the initial fall and the callout time may be measured and timed. One or more algorithms capable of determining the fall event may be manually calibrated for an individual's body measurements, physical condition(s), and/or weight. The algorithm may also be calibrated to detect substantially abrupt and/or a very narrow range of abrupt motions that may indicate a stumble or misstep. The algorithm sequence may be expanded to monitor, collect, transmit, store and analyze timing and sequence of motion or minor motion events for predictive purposes. The algorithm may include capturing of horizontal and rotational motions associated with fall events. Other examples are possible.

In an exemplary embodiment, the device also has a manual alert actuator. It an embodiment, the device 100 may have a recessed button on the inside (or alternately outside) surface of the device 100. This can be a button or switch of any type. The actuator may be imbedded in the bracelet housing to maintain a smooth surface contour. It may be activated upon depression and/or after a pre-set time interval of depression (such as 5 seconds). In other examples the actuator may be triggered on/off by the expansion and contraction of the bracelet's hinged side arms when donning or removing the device. In other examples, the actuator may be exposed on both the inside and outside of the device 100 so that it must be located and squeezed between the thumb and pointer finger of the user from both sides in order to prevent unintentional activation by bumping or normal activity.

In an alternate embodiment, the device may also include a Button that may also be depressed so as to cancel a manual alert inadvertently sent. For example, tapping button after an alert has been sent, may trigger a signal from bracelet 100 to cancel the manual alert. Other examples may include hand waving functionality for cancelling a manually sent signal so as to cancel the manual alert upon detection of a hand waving motion or continued unintentional arm motions by the device 100. Other types of manual alert actuators are possible, including, but not limited to, voice-based actuators.

In another exemplary embodiment, the device 100 may include a feature of waiting period. The waiting period may be a pre-determined period, where there is a pre-set period of time after the fall sensor senses a fall event before the alert is transmitted by transmitter so as to allow for time for a user to manually cancel the alert. The button may be used to manually cancel the fall alert before it is transmitted by transmitter instead of to generate and transmit a cancel signal to the fall response system by transmitter.

In an exemplary embodiment, the device 100 may also be provided with a vibration feature. When a pre-defined vibration pattern is generated then at such instance the user device may send a notification to emergency contact and medical staffs. In an alternate embodiment, on detecting such a specific vibration pattern, the nearby people may also be notified. This may further provide the wearer an opportunity to cancel the alert signal, for example with another press of button, in case the activation was accidental.

In some examples, device 100 may be capable of radiating a high beam light. The beam light may be generated based on the detected emergency by the device that may alert the nearby people.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, apparatus and/or device (e.g., magnetic discs, optical disks, memory, Programmable Logic Devices (PLDs)) used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

Although a few implementations have been described in detail above, other modifications are possible. Moreover, other mechanisms for performing the systems and methods described in this document may be used. In addition, the logic flows depicted in the figures may not require the particular order shown, or sequential order, to achieve desirable results. Other steps may be provided, or steps may be eliminated, from the described flows, and other components may be added to, or removed from, the described systems. Accordingly, other implementations are within the scope of the following claims.

What is claimed is:

1. A wearable medical emergency detection device, the detection device comprising:
a memory unit;
a processor;
an electronic unit comprising a plurality of sensors, wherein the plurality of sensors are placed at plurality of locations and are configured to monitor information of a user; the monitored information includes at least a plurality of vitals of the user;
an information sharing unit configured to transmit the detected information;
an alarming unit for notifying an alarm in case of detected emergency based on the detected information; and
an audio unit configured for receiving one or more voice enabled communications from the notified user to the user wearing the medical emergency detection system.

2. The wearable medical emergency detection device as claimed in claim 1, wherein the detected information is at least a change in position of the user.

3. The wearable medical emergency detection device as claimed in claim 2 further comprising a fall detection unit configured to detect the change in the position of the user; wherein the change is detected by the plurality of sensors.

4. The wearable medical emergency detection device as claimed in claim 1 further comprising a power management module comprising one or more battery units and a plurality of solar enabled charging contacts.

5. The wearable medical emergency detection device as claimed in claim 1, wherein the information is updated based on a triggering of a fall detection of the user.

6. The wearable medical emergency detection device as claimed in claim 1, wherein the memory unit comprises at least the user's personal details, medical history, vitals database, and emergency contact details.

7. The wearable medical emergency detection device as claimed in claim 1 further comprising a display unit for displaying the detected information.

8. The wearable medical emergency detection device as claimed in claim 1 comprising a photo sensor unit to create oximeter via a cross section of the wrist of the user.

9. The wearable medical emergency detection device as claimed in claim 1, wherein the change in position is an abnormal movement of the user or linear acceleration movement.

10. The wearable medical emergency detection device as claimed in claim 1, comprising a transceiver with a wireless communication unit.

11. The wearable medical emergency detection device as claimed in claim 1, further comprising a GPS chipset configured to determine the location of the wearable device and the device capable of transmitting location information with the transceiver.

12. The wearable medical emergency detection device as claimed in claim 1 is a water resistant device.

13. The wearable medical emergency detection device as claimed in claim 1 is further configured to provide a plurality of vibrations to the user in an abnormal detected condition.

14. A wearable medical emergency detection device, the detection device comprising
a memory unit;
a processor configured to trigger an electronic unit; wherein the triggering is based on a change in a position of a user;
the electronic unit comprising a plurality of sensors, wherein the plurality of sensors are configured to monitor a plurality of vitals of a user; wherein the vitals includes at least a blood pressure, a heart rate, an Electrocardiogram reading, a blood oxygen level;
an information sharing unit configured to transmit the monitored plurality of vitals; and
an alarming unit for notifying in case of detected emergency based on an abnormal condition detected based on the transmitted plurality of vitals of the user.

15. A method to detect a medical emergency by a wearable medical emergency detection device, the method comprising steps of:
monitoring an information of a user; wherein the information includes at least vitals of the user;
transmitting to a plurality of other users the monitored information of the user;
notifying in case of detected emergency based on the transmitted information, wherein the notification is communicatively transmitted to a plurality of contacts and;
generating an alarm based on which a plurality of nearby people can help the user under emergency.
